(19) United States
(12) Patent Application Publication
(10) Pub. No.: US 2018/0160884 A1
(43) Pub. Date: Jun. 14, 2018

(54) IMAGING APPARATUS WITH BALLOON AND SHAPED TIP

(71) Applicant: NINEPOINT MEDICAL, INC., Bedford, MA (US)

(72) Inventors: Tsung-Han Tsai, Newton, MA (US); Eman Namati, Concord, MA (US)

(21) Appl. No.: 15/834,348

(22) Filed: Dec. 7, 2017

Related U.S. Application Data
(60) Provisional application No. 62/432,725, filed on Dec. 12, 2016.

(51) Int. Cl. A61B 1/00; A61B 5/00; A61B 1/05; A61B 1/273; A61B 1/267

(52) U.S. Cl. CPC A61B 1/00082; A61B 5/0066; A61B 1/267; A61B 1/273; A61B 1/05

(57) ABSTRACT

Exemplary imaging apparatuses are described. Various imaging apparatuses may implement optical coherence tomography (OCT) and/or optical frequency domain imaging (OFDI) gastrointestinal tissue imaging methods and technologies. Furthermore, the imaging apparatuses may include a rotatable imaging element to scan a bodily lumen, such as the esophagus. The imaging element may be housed within an inflatable balloon. Still further, the imaging apparatuses may include a shaped tip. The shaped tip may aid in guiding an imaging apparatus through the esophagus, or other bodily lumen.

### CROSS-REFERENCE TO RELATED APPLICATIONS

This application claims the benefit of U.S. Provisional Application No. 62/432,725, filed Dec. 12, 2016, which is incorporated herein by reference in its entirety.

### FIELD OF THE DISCLOSURE

The disclosure relates generally to the field of medical imaging technologies, and more particularly to optical imaging probes.

### BACKGROUND OF THE DISCLOSURE

Optical imaging of the interior of the body is often used to assess tissue structures and anatomy. Examples of optical imaging techniques are optical coherence tomography (OCT), angioscopy, near infrared spectroscopy, Raman spectroscopy and fluorescence spectroscopy. Other exemplary methods/procedures include confocal, multi-photon and spectrally-encoded confocal microscopy (SECM).

Typically, devices for optical imaging of the interior of the body include an imaging end (e.g., distal end) functionally coupled to an operating end (e.g., proximal end). The imaging end, which is inserted into the body, is operated and manipulated by the operating end, which is accessible to an external operator.

One example device for optical imaging of the interior of the body is a capsule endoscope. Capsule endoscopes may include an imager, at least one illumination source, and an optical system. Capsule endoscopes may also include other sensors which may sense the in vivo environment, such as temperature sensors, position sensors, motion sensors, pH sensors, and pressure sensors.

Another example device for optical imaging of the interior of the body is a balloon catheter. The balloon catheter may include a delivery system to guide the catheter to a desired location in a lumen of the body. Once positioned in the lumen, an operator may control an operating end of the balloon catheter to cause the balloon to inflate. Similar to the capsule endoscope, an imager may be located within the balloon catheter. The balloon catheter may also include other sensors, such as temperature sensors, pH sensors, position sensors, motion sensors, and pressure sensors.

Current devices for optical imaging of the interior of the body have a number operational drawbacks. For example, capsule endoscopes may be difficult to swallow because of the size of the capsule, and balloon catheters may be difficult to guide through the esophagus without causing undue patient discomfort.

It is with respect to these and other considerations that the present improvements are needed.

### SUMMARY

In view of the forgoing, exemplary imaging apparatuses are described. Various implementations of the imaging apparatuses may implement optical coherence tomography (OCT) and/or optical frequency domain imaging (OFDI) gastrointestinal tissue imaging methods and technologies. Furthermore, the imaging apparatuses may include a rotatable imaging element to scan a bodily lumen, such as the esophagus. The imaging element may be housed within an inflatable balloon. Still further, the imaging apparatuses may include a shaped tip. The shaped tip may aid in guiding an imaging apparatus through the esophagus, or other bodily lumen.

### BRIEF DESCRIPTION OF THE DRAWINGS

By way of example, specific embodiments of the disclosed device will now be described, with reference to the accompanying drawings, in which:

FIG. 1 illustrates a first exemplary imaging apparatus.

FIG. 2 illustrates a second exemplary imaging apparatus.

FIG. 3 illustrates a third exemplary imaging apparatus.

FIG. 4 illustrates a fourth exemplary imaging apparatus.

FIG. 5 illustrates a fifth exemplary imaging apparatus.

FIG. 6 illustrates a sixth exemplary imaging apparatus.

### DETAILED DESCRIPTION

Various examples, implementations, and illustrative configurations are described herein. In some examples, the esophagus is used as an example bodily lumen. However, this is not intended to be limiting. Furthermore, the various depictions are not drawn to scale. Instead, they are drawn in a manner to facilitate understanding. Additionally, the various examples and illustrations can be combined with each other, even where not specifically so stated. Additionally, the described examples are not intended to limit the claims and the present disclosure.

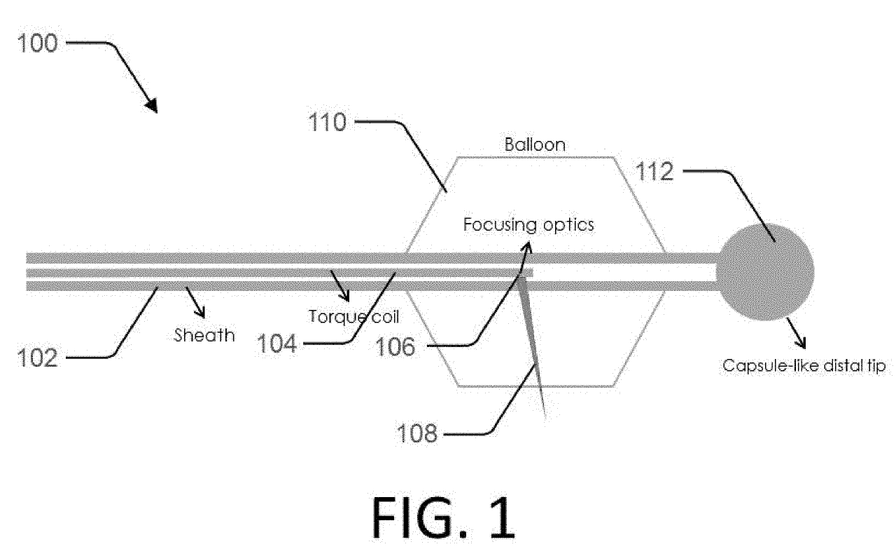

FIG. 1 illustrates a first exemplary imaging apparatus 100. The first exemplary imaging apparatus 100 may implement optical coherence tomography (OCT) and/or optical frequency domain imaging (OFDI) gastrointestinal tissue imaging methods and technologies. The first exemplary imaging apparatus 100 may alternatively implement other scanning optical imaging modalities, such as fluorescence, confocal, or the like. Furthermore, the imaging apparatus 100 may implement other tissue imaging methods and technologies. In one implementation, the first exemplary imaging apparatus 100 may include a system utilizing at least one of OCT or OFDI modalities. Specifically, the first exemplary imaging apparatus 100 may be capable of detecting electromagnetic radiation, such as a back reflected light, from one or more portions associated with tissue. The detected electromagnetic radiation may be processed by the imaging apparatus 100 to ascertain information, such as microstructures, associated with the tissue.

In one implementation, the first exemplary imaging apparatus 100 may include a proximal system including optical fiber, tissue retrieval and treatment functionality, data processing and associated data storage, and the like. As is illustrated in FIG. 1, the imaging apparatus 100 includes a sheath 102. The sheath 102 may be generally associated with a catheter body. The sheath 102 may house a torque coil 104. The torque coil 104 enables rotation of an imaging element 106 associated with the distal portion of the torque coil 104.

The imaging element 106 may be coupled to a fiber optic line. The fiber optic line may be contained or housed within the sheath 102. The fiber optic line may be coupled to a portion of the imaging apparatus 100 that enables OCT and/or OFDI methods and technologies. In one implementation, the fiber optic line may be external of the sheath 102. The imaging element 106 is functional for circumferential scanning by way of at least the rotatable torque coil 104. Helical scanning can also be accomplished by simultaneous rotation and pull back of the torque coil 104.

The imaging element 106 is capable of manipulating, directing and/or focusing an imaging beam 108 onto tissue. Light reflected from the tissue may be processed by the imaging element 106 and conveyed to data processing system(s) associated with the imaging apparatus 100 via the fiber optic line, or the like. In one implementation, the reflected light is conveyed wirelessly to the data processing systems associated with the imaging apparatus 100.

The imaging apparatus 100 may include a balloon 110. The balloon 110 may be inflated via the sheath 102. The balloon 110 may be inflated with air, gas, liquid, or the like. The balloon 110 may be made from a suitable non-compliant, compliant or semi-compliant material such as polyethylene or other polyolefins, polyurethane, flexible polyvinylchloride, Nylon, or the like. An exterior surface of the balloon 110 may be smooth or substantially smooth. Alternatively, the exterior surface of the balloon 110 may be textured with protuberances, or the like, to aid in anchoring the balloon 110 to tissue associated with the esophagus, or the like.

In addition to the balloon 110, the imaging apparatus 100 may include a shaped tip 112 located distal of the imaging apparatus 100. The shaped tip 112 is illustrated as being round or generally circular. In general, however, the shaped tip 112 may be other smooth shapes. For example, the shaped tip 112 may be capsule shaped to include a cylinder with hemispherical ends. That is, the shaped tip 112 may be a spherocylinder. The shaped tip 112 may be solid or hollow. In general, the shaped tip 112 may be made of a polymer, such as polyamides, polyurethanes, nylons, polyethylenes, polyether block amide, polyester, polycarbonate, polypropylene, acrylonitrile-butadiene styrene terpolymer, polyetheretherketone (PEEK), glass, metal, or the like. In various embodiments, the shaped tip 112 can be considered a capsule-like distal tip.

The balloon 110 will be in a deflated state at the time the imaging apparatus 100 is guided through the esophagus, or other bodily lumen. In one implementation, the balloon 110 is tightly bound or wrapped so that there is a uniform circumference or diameter along the length of the sheath 102.

The inclusion of the shaped tip 112 aids in guiding the imaging apparatus 100 through the esophagus, or other bodily lumen. In particular, the inclusion of the shaped tip 112 allows a patient to easily swallow at least a portion of the imaging apparatus 100. In one implementation, the shaped tip 112 aids in guiding the imaging apparatus 100 and the deflated balloon 110 to a desired position within the esophagus. Once a desired position within the esophagus is reached, an operator of the imaging apparatus 100 may cause the balloon 110 to inflate with gas or liquid. The inflated balloon 110 may impart stability, in order to provide a stable imaging environment for the imaging element 106.

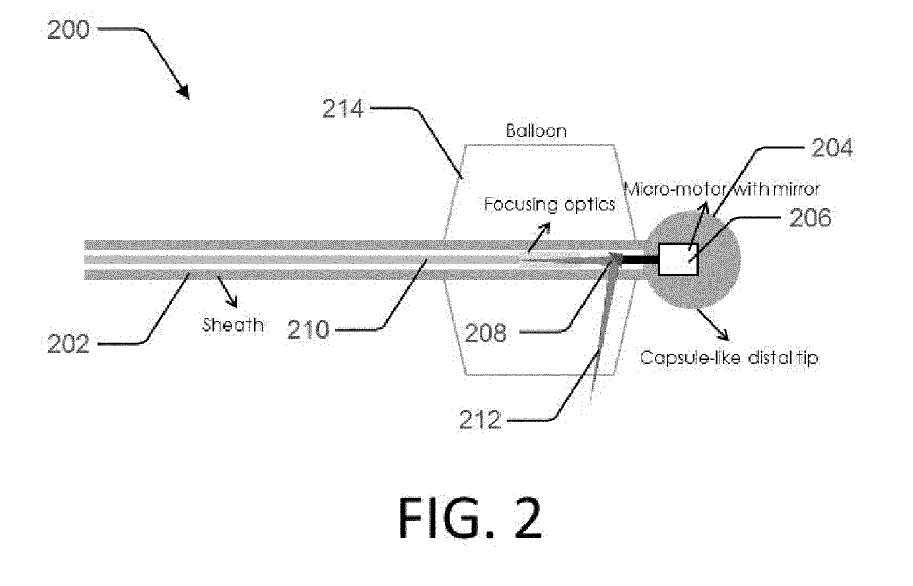

FIG. 2 illustrates a second exemplary imaging apparatus 200. The second exemplary imaging apparatus 200 may implement OCT and/or OFDI gastrointestinal tissue imaging methods and technologies. The second exemplary imaging apparatus 200 may alternatively implement other scanning optical imaging modalities, such as fluorescence, confocal, or the like. Furthermore, the imaging apparatus 200 may implement other tissue imaging methods and technologies. In one implementation, the second exemplary imaging apparatus 200 may include a system utilizing at least one of OCT or OFDI modalities. Specifically, the second exemplary imaging apparatus 200 may be capable of detecting electromagnetic radiation, such as a back reflected light, from one or more portions associated with tissue. The detected electromagnetic radiation may be a processed by the imaging apparatus 200 to ascertain information, such as microstructures, associated with the tissue.

In one implementation, the second exemplary imaging apparatus 200 may include a proximal system including optical fiber, tissue retrieval and treatment functionality, data processing and associated data storage, and the like. As is illustrated in FIG. 2, the imaging apparatus 200 includes a sheath 202. The sheath 202 may be generally associated with a catheter body.

The imaging apparatus 200 may include a shaped tip 204 located distal of the imaging apparatus 200. The shaped tip 204 is illustrated as being round or generally circular. In general, however, the shaped tip 204 may be other smooth shapes. For example, the shaped tip 204 may be capsule shaped to include a cylinder with hemispherical ends. That is, the shaped tip 204 may be a spherocylinder. In general, the shaped tip 204 may be made of a polymer, such as polyamides, polyurethanes, nylons, polyethylenes, polyether block amide, polyester, polycarbonate, polypropylene, acrylonitrile-butadiene styrene terpolymer, polyetheretherketone (PEEK), glass, metal, or the like.

The shaped tip 204 may house a motor 206. In one implementation, the motor 206 is a micro motor. The motor 206 may include a mirror. The motor 206 enables rotation of an imaging element 208. The motor 206 may be wirelessly or wireline controlled at a proximate end of the imaging apparatus 200. In particular, an operator of the imaging apparatus 200 may control the functionality of the motor 206.

The imaging element 208 may be coupled to a fiber optic line 210. The fiber optic line 210 may be contained or housed within the sheath 202. The fiber optic line 210 may be coupled to a portion of the imaging apparatus 200 that enables OCT and/or OFDI methods and technologies. In one implementation, the fiber optic line 210 may be external of the sheath 202. The imaging element 208 is functional for rotary scanning by way of at least the motor 206.

The imaging element 208 is capable of manipulating, directing and/or focusing an imaging beam 212 onto tissue. Light reflected from the tissue may be processed by the imaging element 208 and conveyed to data processing system(s) associated with the imaging apparatus 200 via the fiber optic line, or the like. In one implementation, the reflected light is conveyed wirelessly to the data processing systems associated with the imaging apparatus 200.

The imaging apparatus 200 may include a balloon 214. The balloon 214 may be inflated via the sheath 202. The balloon 214 may be inflated with air, gas, liquid, or the like. The balloon 214 may be made from a suitable compliant or semi-compliant material such as polyethylene or other polyolefins, polyurethane, flexible polyvinylchloride, Nylon, or the like. An exterior surface of the balloon 214 may be smooth or substantially smooth. Alternatively, the exterior surface of the balloon 214 may be textured with protuberances, or the like, to aid in anchoring the balloon 214 to tissue associated with the esophagus, or the like.

The balloon 214 will be in a deflated state at the time the imaging apparatus 200 is guided through the esophagus, or other bodily lumen. In one implementation, the balloon 214 is tightly bound or wrapped so that there is a uniform circumference or diameter along the length of the sheath 202.

The inclusion of the shaped tip 204 aids in guiding the imaging apparatus 200 through the esophagus, or other bodily lumen. In particular, the inclusion of the shaped tip 204 allows a patient to easily swallow at least a portion of the imaging apparatus 200. In one implementation, the shaped tip 204 aids in guiding the imaging apparatus 200 and the deflated balloon 214 to a desired position within the esophagus. Once a desired position within the esophagus is reached, an operator of the imaging apparatus 200 may cause the balloon 214 to inflate with gas or liquid. The inflated balloon 214 may impart stability, in order to provide a stable imaging environment for the imaging element 106.

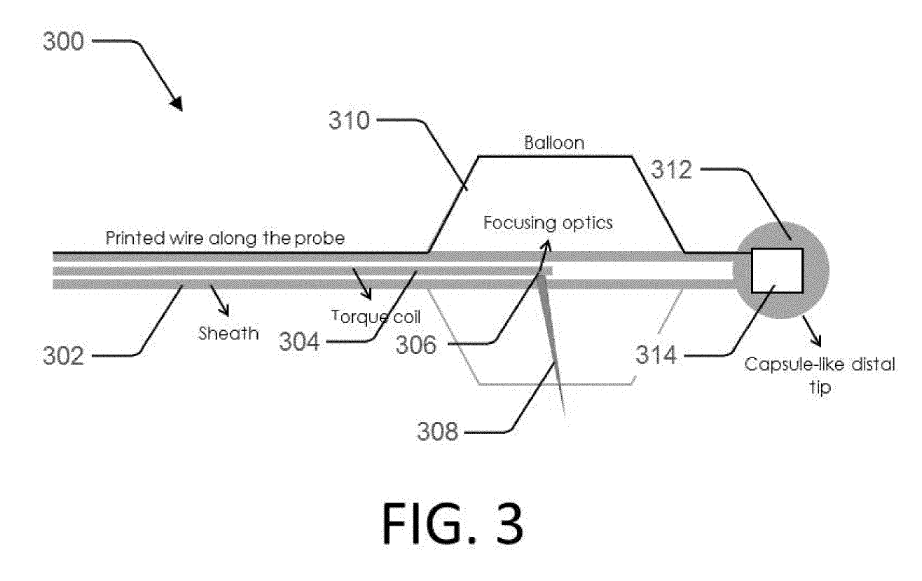

FIG. 3 illustrates a third exemplary imaging apparatus 300. The third exemplary imaging apparatus 300 may implement OCT and/or OFDI gastrointestinal tissue imaging methods and technologies. The third exemplary imaging apparatus 100 may alternatively implement other scanning optical imaging modalities, such as fluorescence, confocal, or the like. Furthermore, the imaging apparatus 300 may implement other tissue imaging methods and technologies. In one implementation, the third exemplary imaging apparatus 300 may include a system utilizing at least one of OCT or OFDI modalities. Specifically, the third exemplary imaging apparatus 300 may be capable of detecting electromagnetic radiation, such as a back reflected light, from one or more portions associated with tissue. The detected electromagnetic radiation may be a processed by the imaging apparatus 300 to ascertain information, such as microstructures, associated with the tissue.

In one implementation, the third exemplary imaging apparatus 300 may include a proximal system including optical fiber, tissue retrieval and treatment functionality, data processing and associated data storage, and the like. As is illustrated in FIG. 3, the imaging apparatus 300 includes a sheath 302. The sheath 302 may be generally associated with a catheter body. The sheath 302 may house a torque coil 304. The torque coil 304 enables rotation of an imaging element 306 associated with the distal portion of the torque coil 304.

The imaging element 306 may be coupled to a fiber optic line. The fiber optic line may be contained or housed within the sheath 302. The fiber optic line may be coupled to a portion of the imaging apparatus 300 that enables OCT and/or OFDI methods and technologies. In one implementation, the fiber optic line may be external of the sheath 302. The imaging element 306 is functional for helical scanning by way of at least the rotatable torque coil 304.

The imaging element 306 is capable of focusing, manipulating, and/or directing an imaging beam 308 onto tissue. Light reflected from the tissue may be processed by the imaging element 306 and conveyed to data processing system(s) associated with the imaging apparatus 300 via the fiber optic line, or the like. In one implementation, the reflected light is conveyed wirelessly to the data processing systems associated with the imaging apparatus 300.

The imaging apparatus 300 may include a balloon 310. The balloon 310 may be inflated via the sheath 302. The balloon 310 may be inflated with air, gas, liquid, or the like. The balloon 310 may be made from a suitable compliant or semi-compliant material such as polyethylene or other polyolefins, polyurethane, flexible polyvinylchloride, Nylon, or the like. An exterior surface of the balloon 310 may be smooth or substantially smooth. Alternatively, the exterior surface of the balloon 310 may be textured with protuberances, or the like, to aid in anchoring the balloon 310 to tissue associated with the esophagus, or the like.

In addition to the balloon 310, the imaging apparatus 300 may include a shaped tip 312 located distal of the imaging apparatus 300. The shaped tip 312 is illustrated as being round or generally circular. In general, however, the shaped tip 312 may be other smooth shapes. For example, the shaped tip 312 may be capsule shaped to include a cylinder with hemispherical ends. That is, the shaped tip 312 may be a spherocylinder. In general, the shaped tip 312 may be made of a polymer, such as polyamides, polyurethanes, nylons, polyethylenes, polyether block amide, polyester, polycarbonate, polypropylene, acrylonitrile-butadiene styrene terpolymer, polyetheretherketone (PEEK), glass, metal, or the like.

A wired device 314 may be housed within the shaped tip 312. The wire device 314 may be coupled to a proximate end of the imaging apparatus 300 by way of at least one wire 316. The at least one wire 316 may be disposed on an exterior surface of the sheath 302. Furthermore, the at least one wire 316 may be disposed on an exterior surface of the balloon 310. The wire device 314 may provide a pressure sensor for monitoring a pressure applied to the shaped tip 312, producing a pressure signal indicative of the pressure. Also, the wire device 314 may provide a location sensor for monitoring a location of the shaped tip 312 within a bodily lumen, relative to a reference point. As another option, the wire device 314 may provide a chemical sensor for monitoring at least one chemical parameter from within a bodily lumen, such as pH. As a still further function, the wire device 314 may provide functionality for performing a tissue retrieval, for example, to take a tissue sample at a desired site within a bodily lumen. In one implementation, information obtained by the wire device 314 is conveyed, via the at least one wire 316, to the data processing systems associated with the imaging apparatus 300.

In one implementation, the wire device 314 is a wireless device. In such an implementation, the at least one wire 316 may not be necessary. The wire device 314 implemented as a wireless device may communicate wirelessly with the data processing systems associated with the imaging apparatus 300.

The balloon 310 will be in a deflated state at the time the imaging apparatus 300 is guided through the esophagus, or other bodily lumen. In one implementation, the balloon 310 is tightly bound or wrapped so that there is a uniform circumference or diameter along the length of the sheath 302.

The inclusion of the shaped tip 312 aids in guiding the imaging apparatus 300 through the esophagus, or other bodily lumen. In particular, the inclusion of the shaped tip 312 allows a patient to easily swallow at least a portion of the imaging apparatus 300. In one implementation, the shaped tip 312 aids in guiding the imaging apparatus 300 and the deflated balloon 310 to a desired position within the esophagus. Once a desired position within the esophagus is reached, an operator of the imaging apparatus 300 may cause the balloon 310 to inflate with gas or liquid. The inflated balloon 310 may impart stability, in order to provide a stable imaging environment for the imaging element 306.

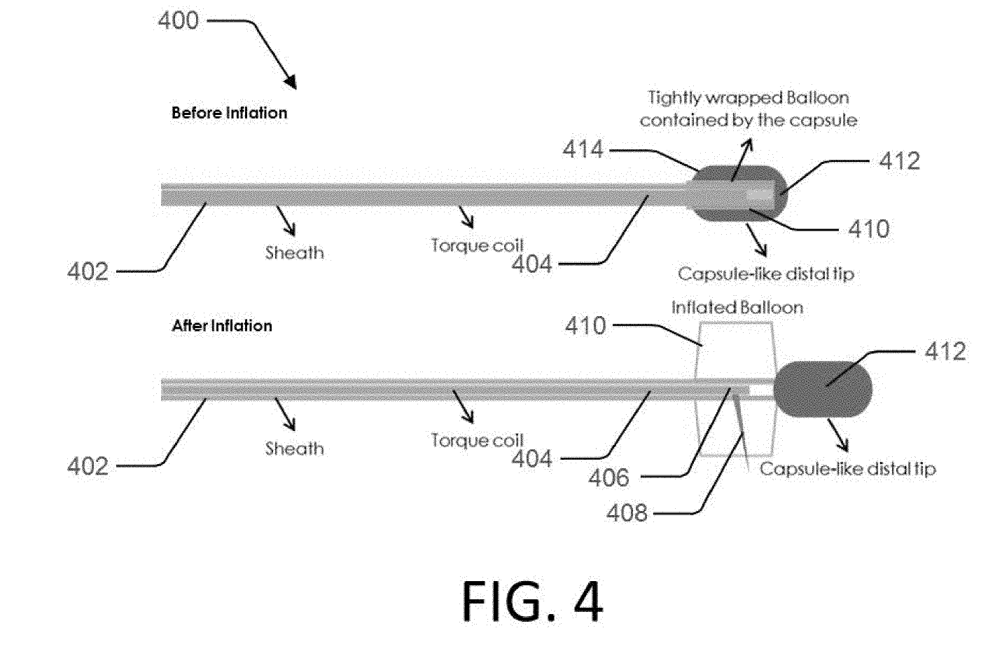

FIG. 4 illustrates a fourth exemplary imaging apparatus 400. The fourth exemplary imaging apparatus 400 may implement OCT and/or OFDI gastrointestinal tissue imaging methods and technologies. The fourth exemplary imaging apparatus 400 may alternatively implement other scanning optical imaging modalities, such as fluorescence, confocal, or the like. Furthermore, the imaging apparatus 400 may implement other tissue imaging methods and technologies. In one implementation, the fourth exemplary imaging apparatus 400 may include a system utilizing at least one of OCT or OFDI modalities. Specifically, the fourth exemplary imaging apparatus 400 may be capable of detecting electromagnetic radiation, such as a back reflected light, from one or more portions associated with tissue. The detected electromagnetic radiation may be a processed by the imaging apparatus 400 to ascertain information, such as microstructures, associated with the tissue.

In one implementation, the fourth exemplary imaging apparatus 400 may include a proximal system including optical fiber, tissue retrieval and treatment functionality, data processing and associated data storage, and the like. As is illustrated in FIG. 4, the imaging apparatus 400 includes a sheath 402. The sheath 402 may be generally associated with a catheter body. The sheath 402 may house a torque coil 404. The torque coil 404 enables rotation of an imaging element 406 associated with the distal portion of the torque coil 404. In another embodiment, rotation of the imaging element 406 is achieved by way of a motor, such as a micro motor.

The imaging element 406 may be coupled to a fiber optic line. The fiber optic line may be contained or housed within the sheath 402. The fiber optic line may be coupled to a portion of the imaging apparatus 400 that enables OCT and/or OFDI methods and technologies. In one implementation, the fiber optic line may be external of the sheath 402. The imaging element 406 is functional for helical scanning by way of at least the rotatable torque coil 404.

The imaging element 406 is capable of focusing, manipulating, and/or directing an imaging beam 408 onto tissue. Light reflected from the tissue may be processed by the imaging element 406 and conveyed to data processing system(s) associated with the imaging apparatus 400 via the fiber optic line, or the like. In one implementation, the reflected light is conveyed wirelessly to the data processing systems associated with the imaging apparatus 400.

The imaging apparatus 400 may include a balloon 410. The balloon 410 may be inflated via the sheath 402. The balloon 410 may be inflated with air, gas, liquid, or the like. The balloon 410 may be made from a suitable compliant or semi-compliant material such as polyethylene or other polyolefins, polyurethane, flexible polyvinylchloride, Nylon, or the like. An exterior surface of the balloon 410 may be smooth or substantially smooth. Alternatively, the exterior surface of the balloon 410 may be textured with protuberances, or the like, to aid in anchoring the balloon 410 to tissue associated with the esophagus, or the like.

In addition to the balloon 410, the imaging apparatus 400 may include a shaped tip 412 located distal of the imaging apparatus 400. The shaped tip 412 is illustrated as being round or generally spherocylinder. In general, however, the shaped tip 412 may be other smooth shapes. In general, the shaped tip 412 may be made of a polymer, such as polyamides, polyurethanes, nylons, polyethylenes, polyether block amide, polyester, polycarbonate, polypropylene, acrylonitrile-butadiene styrene terpolymer, polyetheretherketone (PEEK), glass, metal, or the like.

As is illustrated, the balloon 410 may be housed within the shaped tip 412. Specifically, the balloon 410 may be housed within the shaped tip 412 while it is in a deflated state. The balloon 410 will be in a deflated state at the time the imaging apparatus 400 is guided through the esophagus, or other bodily lumen.

The inclusion of the shaped tip 412 aids in guiding the imaging apparatus 400 through the esophagus, or other bodily lumen. In particular, the inclusion of the shaped tip 412 allows a patient to easily swallow at least a portion of the imaging apparatus 400. In one implementation, the shaped tip 412 aids in guiding the imaging apparatus 400 and the deflated balloon 410 to a desired position within the esophagus. Once a desired position within the esophagus is reached, an operator of the imaging apparatus 400 may cause the balloon 410 to inflate with gas or liquid. The inflated balloon 410 may impart stability, in order to provide a stable imaging environment for the imaging element 406. The process of inflating the balloon 410 includes causing the balloon 410, in a deflated state and while the balloon 410 is inflating, to exit the shaped tip 412 at an opening 414 in the shaped tip 412.

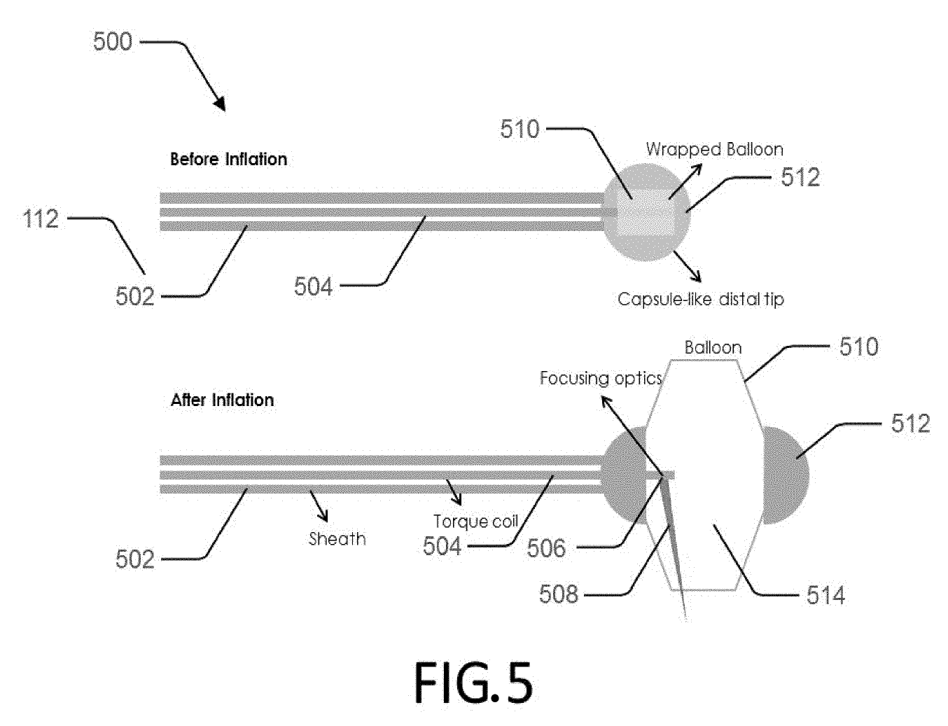

FIG. 5 illustrates a fifth exemplary imaging apparatus 500. The fifth exemplary imaging apparatus 500 may implement OCT and/or OFDI gastrointestinal tissue imaging methods and technologies. The fifth exemplary imaging apparatus 500 may alternatively implement other scanning optical imaging modalities, such as fluorescence, confocal, or the like. Furthermore, the imaging apparatus 500 may implement other tissue imaging methods and technologies. In one implementation, the fifth exemplary imaging apparatus 500 may include a system utilizing at least one of OCT or OFDI modalities. Specifically, the fifth exemplary imaging apparatus 500 may be capable of detecting electromagnetic radiation, such as a back reflected light, from one or more portions associated with tissue. The detected electromagnetic radiation may be a processed by the imaging apparatus 500 to ascertain information, such as microstructures, associated with the tissue.

In one implementation, the fifth exemplary imaging apparatus 500 may include a proximal system including optical fiber, tissue retrieval and treatment functionality, data processing and associated data storage, and the like. As is illustrated in FIG. 5, the imaging apparatus 500 includes a sheath 502. The sheath 502 may be generally associated with a catheter body. The sheath 502 may house a torque coil 504. The torque coil 504 enables rotation of an imaging element 506 associated with the distal portion of the torque coil 504. In another embodiment, rotation of the imaging element 506 is achieved by way of a motor, such as a micro motor.

The imaging element 506 may be coupled to a fiber optic line. The fiber optic line may be contained or housed within the sheath 502. The fiber optic line may be coupled to a portion of the imaging apparatus 500 that enables OCT and/or OFDI methods and technologies. In one implementation, the fiber optic line may be external of the sheath 502. The imaging element 506 is functional for helical scanning by way of at least the rotatable torque coil 504.

The imaging element 506 is capable of focusing, manipulating, and/or directing an imaging beam 508 onto tissue. Light reflected from the tissue may be processed by the imaging element 506 and conveyed to data processing system(s) associated with the imaging apparatus 500 via the fiber optic line, or the like. In one implementation, the reflected light is conveyed wirelessly to the data processing systems associated with the imaging apparatus 500.

The imaging apparatus 500 may include a balloon 510. The balloon 510 may be inflated via the sheath 502. The balloon 510 may be inflated with air, gas, liquid, or the like. The balloon 510 may be made from a suitable compliant or semi-compliant material such as polyethylene or other polyolefins, polyurethane, flexible polyvinylchloride, Nylon, or the like. An exterior surface of the balloon 510 may be smooth or substantially smooth. Alternatively, the exterior surface of the balloon 510 may be textured with protuberances, or the like, to aid in anchoring the balloon 510 to tissue associated with the esophagus, or the like.

In addition to the balloon 510, the imaging apparatus 500 may include a shaped tip 512 located distal of the imaging apparatus 500. The shaped tip 512 is illustrated as being round or generally spherocylinder. In general, however, the shaped tip 512 may be other smooth shapes. In general, the shaped tip 512 may be made of a polymer, such as polyamides, polyurethanes, nylons, polyethylenes, polyether block amide, polyester, polycarbonate, polypropylene, acrylonitrile-butadiene styrene terpolymer, polyetheretherketone (PEEK), glass, metal, or the like.

As is illustrated, the balloon 510 may be housed within the shaped tip 512. Specifically, the balloon 510 may be housed within the shaped tip 512 while it is in a deflated state. The balloon 510 will be in a deflated state at the time the imaging apparatus 500 is guided through the esophagus, or other bodily lumen.

The inclusion of the shaped tip 512 aids in guiding the imaging apparatus 500 through the esophagus, or other bodily lumen. In particular, the inclusion of the shaped tip 512 allows a patient to easily swallow at least a portion of the imaging apparatus 500. In one implementation, the shaped tip 512 aids in guiding the imaging apparatus 500 and the deflated balloon 510 to a desired position within the esophagus. Once a desired position within the esophagus is reached, an operator of the imaging apparatus 500 may cause the balloon 510 to inflate with gas or liquid. The inflated balloon 510 may impart stability, in order to provide a stable imaging environment for the imaging element 506. The process of inflating the balloon 510 includes causing the balloon 510, in a deflated state and while the balloon 510 is inflating, to exit the shaped tip 512 at an opening 514 in the shaped tip 512. More particularly, as the balloon 510 inflates, the shaped tip 512 splits at an aperture associated with the shaped tip 512.

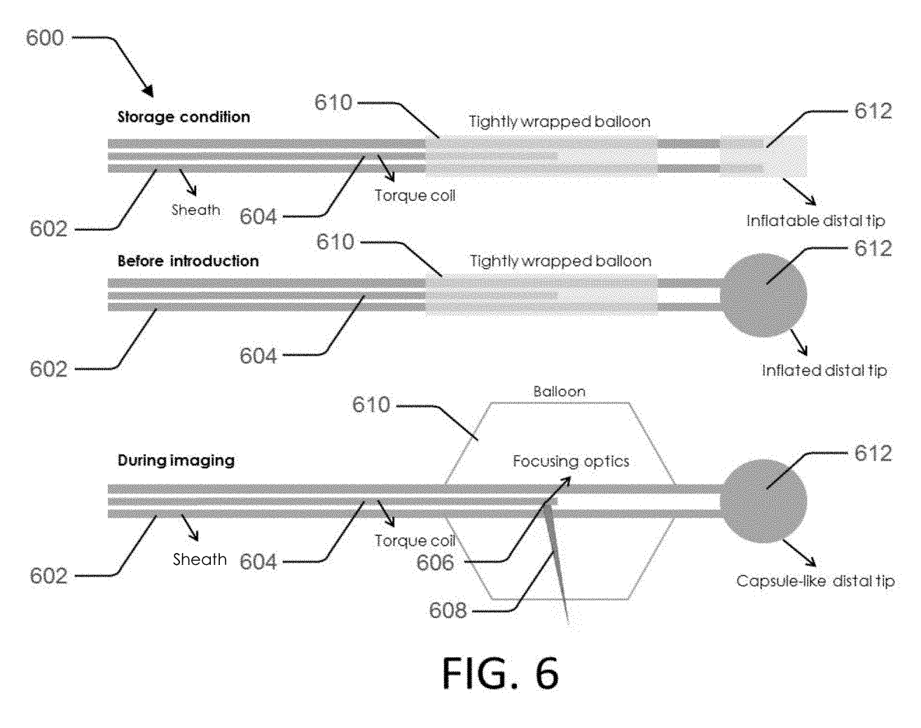

FIG. 6 illustrates a sixth exemplary imaging apparatus 600. The sixth exemplary imaging apparatus 600 may implement OCT and/or OFDI gastrointestinal tissue imaging methods and technologies. The sixth exemplary imaging apparatus 600 may alternatively implement other scanning optical imaging modalities, such as fluorescence, confocal, or the like. Furthermore, the imaging apparatus 600 may implement other tissue imaging methods and technologies. In one implementation, the sixth exemplary imaging apparatus 600 may include a system utilizing at least one of OCT or OFDI modalities. Specifically, the sixth exemplary imaging apparatus 600 may be capable of detecting electromagnetic radiation, such as a back reflected light, from one or more portions associated with tissue. The detected electromagnetic radiation may be a processed by the imaging apparatus 600 to ascertain information, such as microstructures, associated with the tissue.

In one implementation, the sixth exemplary imaging apparatus 600 may include a proximal system including optical fiber, tissue retrieval and treatment functionality, data processing and associated data storage, and the like. As is illustrated in FIG. 6, the imaging apparatus 600 includes a sheath 602. The sheath 602 to may be generally associated with a catheter body. The sheath 602 may house a torque coil 604. The torque coil 604 enables rotation of an imaging element 606 associated with the distal portion of the torque coil 604.

The imaging element 606 may be coupled to a fiber optic line. The fiber optic line may be contained or housed within the sheath 602. The fiber optic line may be coupled to a portion of the imaging apparatus 600 that enables OCT and/or OFDI methods and technologies. In one implementation, the fiber optic line may be external of the sheath 602. The imaging element 606 is functional for helical scanning by way of at least the rotatable torque coil 604.

The imaging element 606 is capable of focusing, manipulating, and/or directing an imaging beam 608 onto tissue. Light reflected from the tissue may be processed by the imaging element 606 and conveyed to data processing system(s) associated with the imaging apparatus 600 via the fiber optic line, or the like. In one implementation, the reflected light is conveyed wirelessly to the data processing systems associated with the imaging apparatus 600.

The imaging apparatus 600 may include a balloon 610. The balloon 610 may be inflated via the sheath 602. The balloon 610 may be inflated with air, gas, liquid, or the like. The balloon 610 may be made from a suitable compliant or semi-compliant material such as polyethylene or other polyolefins, polyurethane, flexible polyvinylchloride, Nylon, or the like. An exterior surface of the balloon 610 may be smooth or substantially smooth. Alternatively, the exterior surface of the balloon 610 may be textured with protuberances, or the like, to aid in anchoring the balloon 610 to tissue associated with the esophagus, or the like.

In addition to the balloon 610, the imaging apparatus 600 may include a shaped tip 612 located distal of the imaging apparatus 600. The shaped tip 612 is illustrated as being round or generally circular. In general, however, the shaped tip 612 may be other smooth shapes. For example, the shaped tip 612 may be capsule shaped to include a cylinder with hemispherical ends. That is, the shaped tip 612 may be a spherocylinder. The shaped tip 612 may be inflatable. Specifically, the shaped tip 612 may be inflated with air, gas, liquid, or the like, before insertion in the esophagus, or other bodily lumen. In general, the shaped tip 612 may be made from a suitable compliant or semi-compliant material such as polyethylene or other polyolefins, polyurethane, flexible polyvinylchloride, Nylon, or the like.

The balloon 610 will be in a deflated state at the time the imaging apparatus 600 is guided through the esophagus, or other bodily lumen. In one implementation, the balloon 610 is loosely or lightly bound are wrapped during storage. In one implementation, prior to insertion in a bodily lumen, the balloon 610 is caused to be tightly bound or wrapped so that there is a uniform circumference or diameter along the length of the sheath 602.

The inclusion of the shaped tip 612 aids in guiding the imaging apparatus 600 through the esophagus, or other bodily lumen. In particular, the inclusion of the shaped tip 612 allows a patient to easily swallow at least a portion of the imaging apparatus 600. In one implementation, the shaped tip 612 aids in guiding the imaging apparatus 600 and the deflated balloon 610 to a desired position within the esophagus. Once a desired position within the esophagus is reached, an operator of the imaging apparatus 600 may cause the balloon 610 to inflate with gas or liquid. The inflated balloon 610 may impart stability, in order to provide a stable imaging environment for the imaging element 606.

In various embodiments, the shaped tips described herein and depicted in FIGS. 1-6 can be considered capsule-like distal tips.

The embodiments have been described and illustrated as including various structures, elements, and operational functionalities. Those described various structures, elements, and operational functionalities may apply to and be used with each of the embodiments described herein.

Furthermore, while imaging apparatuses have been described with reference to certain embodiments, it will be understood by those skilled in the art that various changes may be made and equivalents may be substituted without departing from the spirit and scope of the claims of the application. Other modifications may be made to adapt a particular situation or material to the teachings disclosed above without departing from the scope of the claims. Therefore, the claims should not be construed as being limited to any one of the particular embodiments disclosed, but to any embodiments that fall within the scope of the claims.

1. An imaging apparatus, comprising:
a sheath;
an imaging element disposed in the sheath;
a shaped tip disposed at an end of the sheath;
an inflatable balloon associated with the sheath, the inflatable balloon being inflatable via the sheath.

2. The imaging apparatus according to claim 1, wherein the inflatable balloon surrounds a portion of the sheath and is adjacent the shaped tip.

3. The imaging apparatus according to claim 1, wherein the shaped tip has a smooth exterior surface.

4. The imaging apparatus according to claim 1, wherein the shaped tip is round.

5. The imaging apparatus according to claim 1, wherein the imaging element is disposed in a portion of the sheath associated with the inflatable balloon.

6. The imaging apparatus according to claim 1, further comprising a torque coil coupled to the imaging element, the torque coil to rotate the imaging element.

7. The imaging apparatus according to claim 1, wherein the shaped tip includes a motor housed in the shaped tip, the motor to rotate the imaging element.

8. The imaging apparatus according to claim 7, wherein the motor is wirelessly or wireline controllable.

9. An imaging apparatus, comprising:
a sheath;
an imaging element disposed in the sheath;
a shaped tip disposed at an end of the sheath;
an inflatable balloon disposed in the shaped tip, the inflatable balloon being inflatable via the sheath.

10. The imaging apparatus according to claim 9, wherein the shaped tip has a smooth exterior surface.

11. The imaging apparatus according to claim 9, wherein the shaped tip is a cylinder with hemispherical ends.

12. The imaging apparatus according to claim 9, further comprising a torque coil coupled to the imaging element, the torque coil to rotate the imaging element.

13. The imaging apparatus according to claim 9, wherein the shaped tip includes an opening to allow release of the inflatable balloon.

14. An imaging apparatus, comprising:
a sheath;
an imaging element disposed in the sheath;
an inflatable shaped tip disposed at an end of the sheath, the inflatable shaped tip being inflatable via the sheath;
an inflatable balloon associated with the sheath, the inflatable balloon being inflatable via the sheath.

15. The imaging apparatus according to claim 14, wherein the inflatable balloon surrounds a portion of the sheath and is adjacent the inflatable shaped tip.

16. The imaging apparatus according to claim 14, wherein the shaped tip has a smooth exterior surface in an inflated state.

17. The imaging apparatus according to claim 14, wherein the shaped tip is round in an inflated state.

18. The imaging apparatus according to claim 14, wherein the imaging element is disposed in a portion of the sheath associated with the inflatable balloon.

19. The imaging apparatus according to claim 14, further comprising a torque coil coupled to the imaging element, the torque coil to rotate the imaging element.
